(19) United States
(12) Patent Application Publication
(10) Pub. No.: US 2005/0070932 A1
(43) Pub. Date: Mar. 31, 2005

(54) Deep fascia anchors

(76) Inventor: Mark H. Falahee, Ann Arbor, MI (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/950,990

(22) Filed: Sep. 27, 2004

Related U.S. Application Data
(60) Provisional application No. 60/506,089, filed on Sep. 25, 2003.

(51) Int. Cl. A61B 17/08

(52) U.S. Cl. 606/151

(57) ABSTRACT

Deep fascia anchors provide a convenient way to bring fascia back to its anatomic location, thereby avoiding many of the problems and complications with current approaches. In the preferred embodiment, the anchors are made of a silastic or biodegradable material utilizing a design which mimics the spinous process. The anchors may attach to any suitable form of instrumentation, including rods, plates, and so forth. The anchors are adjustable to suit different mechanical structures. Anchors according to the invention include two opposing flexible arms which wrap around and lock on to the instrumentation, with a center tip portion preferably including attachment points or holes to which the deep fascia may be attached, much like the natural spinous process. Multiple anchors may be used according to the invention as dictated by the length of the instrumentation, and varying sizes and shapes may be provided to mimic anatomic differences in location (i.e., cervical, thoracic, lumbar) and patient (i.e., child, adolescent, adult, male/female).

### REFERENCE TO RELATED APPLICATION

This application claims priority from U.S. Provisional Patent Application Ser. No. 60/506,089, filed Sep. 25, 2003, the entire content of which is incorporated herein by reference.

### FIELD OF THE INVENTION

This invention relates generally to surgical procedures and, in particular, to deep fascia anchors particularly suited to certain spinal surgical approaches.

### BACKGROUND OF THE INVENTION

Deep fascia is released from spinous processes in the course of surgical dissection for approaches to the spine for the purpose of implementing spinal fusions and instrumentations. Typically, the spinous processes are removed to do decompressive work around the nerves, leaving a large void, and no place to reattach the deep fascia.

FIG. 1 is a drawing which shows a skin incision at 102, revealing deep fascia 104 attached to the tips of the spinous processes 106. FIG. 2 is a cross section, showing a vertebral body at 110, a spinous process at 106, and muscle extending between the spinous process and deep fascia 104.

FIG. 3 is a drawing which shows the way in which the deep fascia are released, and spinous processes moved for decompression. Instrumentation, such as a rod and screw fixation system 310, is in place on either side of the spinal cord 302. FIG. 4 is a drawing which shows closure of the deep fascia over a drainage tube 410 utilizing sutures or staples 406. As there is currently no anatomic replacement for the spinous processes, the fascia edges are typically sewn together, over drainage tube(s), leaving a large void 402.

These voids left through existing techniques may lead to various complications. The creation of a large pocket results in sections prone to the development of hematomas. These areas are also a rich culture medium for bacteria, increasing wound infection and breakdown. This, in turn, may cause increased wound swelling, deforming the fascia layers. All of the above slows the healing process, which requires replacement of hematoma with scar tissue. The increase in scar tissue, in turn, may complicate subsequent surgical procedures. Additionally, if the fascia is not returned to its anatomic location, it is often placed in an infolded position curving over the spine muscles, and down into the spinous processes. This may be a source of back muscle fatigue, and spasm, which often occur postoperatively. Cosmetic and functional problems are possible as well.

### SUMMARY OF THE INVENTION

This invention broadly resides in deep fascia anchors that provide a convenient way to bring fascia back to its anatomic location, thereby avoiding many of the problems and complications with current approaches. In the preferred embodiment, the anchors are made of a silastic or other biocompatible or biodegradable material utilizing a design which mimics the spinous process. The anchors may attach to any suitable form of instrumentation, including rods, plates, and so forth. The anchors are adjustable to suit different mechanical structures.

Anchors according to the invention include two opposing flexible arms which wrap around and lock on to the instrumentation, with a center tip portion preferably including attachment points or holes to which the deep fascia may be attached, much like the natural spinous process. Multiple anchors may be used according to the invention as dictated by the length of the instrumentation, and varying sizes and shapes may be provided to mimic anatomic differences in location (i.e., cervical, thoracic, lumbar) and patient (i.e., child, adolescent, adult, male/female).

### BRIEF DESCRIPTION OF THE DRAWINGS

FIG. 1 is a drawing which shows deep fascia attached to spinous processes;

FIG. 2 is a cross section of the situation of FIG. 1;

FIG. 3 is a drawing which shows fascia released, spinous processes removed for decompression, and instrumentation in place;

FIG. 4 is a drawing which illustrates closure of deep fascia over a drainage tube;

FIG. 5 is a perspective drawing of a preferred embodiment of the invention;

FIG. 6 is a perspective drawing showing two of the anchors of FIG. 5 in position with respect to opposing rod and screw fixation assemblies;

FIG. 7A is a close-up view of one attachment mechanism according to the invention;

FIG. 7B is a close-up view of an alternative attachment mechanism according to the invention;

FIG. 8 is a drawing which shows reattaching deep fascia to the inventive anchors, significantly reducing “dead space,” and restoring normal anatomic relationship to the spine and spinous muscles;

FIG. 9 is a drawing of an alternative embodiment of the invention, including a central section which is, itself, formed by passing a strip through itself; and

FIG. 10 is a close-up view of the loop created using the embodiment of FIG. 9.

### DETAILED DESCRIPTION OF THE INVENTION

Having discussed the prior art with respect to FIGS. 1-4, the reader's attention is now directed to FIG. 5, which shows a preferred deep fascia anchor according to the invention. Broadly, the anchor includes two side arms with locking mechanisms to create loops 502, 504, which may be adjustably placed around different instrumentation, including rods, plates, screws, and so forth. Between these side arms, there is a section 506 to which deep fascia may be attached. In the preferred embodiment, holes 508 are provided for this purpose, though this is not necessary to the invention, in that the material of the anchor may be such that sutures may pass through without the need for apertures. The profile of the anchor, indicated by the double-headed arrow, is sufficiently low that the smaller profile and lower contour allows for an effective yet cosmetic solution to the problem set forth in the Background of the Invention, including in the cervical and thoracic regions of the spine.

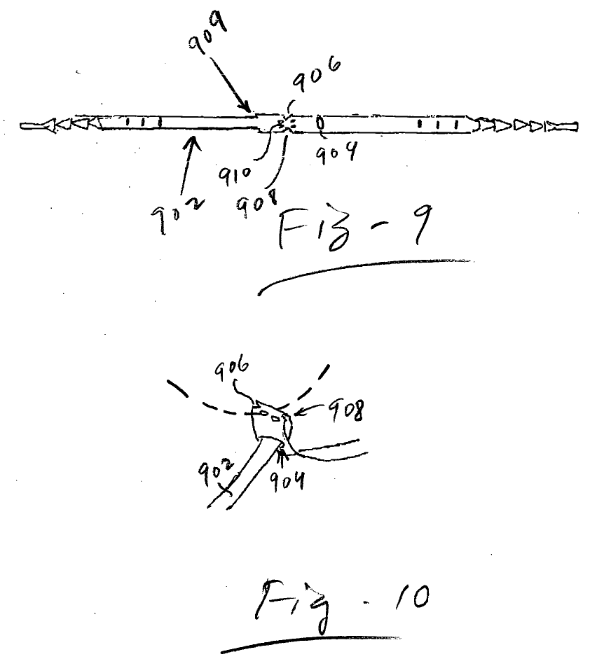

FIG. 6 is a drawing which shows two of the anchors of FIG. 5 in place around two opposing rods 310. The side arms have been looped around these rods, and locked into position, with the excess of the loop material, if any, 610, 612, being available for trimming. FIG. 7A is a close-up view drawing of one locking mechanism, wherein the end of the side arm 702 includes a plurality of barbs which fit through one or more apertures 704 in the side arm, much like plastic ties used for bags and other closures. FIG. 7B is a close-up view drawing of an alternative locking mechanism, wherein beads along the side arm 710 fit into one or more holes 712 with slots once the material is stretched. Although two alternative mechanisms are shown in FIGS. 7A and 7B, it will be apparent to one of skill in the art that any other type of locking mechanism may be used, so long as it provides sufficient adjustability and functionality with respect to the applications described herein.

FIG. 8 is a drawing which shows the reattachment of deep fascia to anchors according to the invention, significantly reducing dead spaces, and restoring normal anatomic relationship to the spine and spinous processes. The sutures 802 are progressively attached to sections 506 of the anchors, thereby closing the wound in a much more controlled and anatomically correct manner.

FIG. 9 is a drawing of an alternative embodiment of the invention, including a central section which is, itself, formed by passing the strip 902 through slot 904, allowing a strip of consistent thickness to be used. Optional indents 906, 908 may be provided for folding along with an optional “stop point” 909, creating an artificial spinous process having a height preferably in the range of 6-10 mm. Suture holes are shown at 910, preferably near the top of the structure so the facia comes together and seals the wound without macropore material sticking out. FIG. 10 is a close-up view of the loop created using the embodiment of FIG. 9, with a suture being indicated with the broken line.

Regardless of embodiment, varying sizes and shapes may be provided to mimic anatomic differences in location (i.e., cervical, thoracic, lumbar) and patient (i.e., child, adolescent, adult, male/female). For example, three basic sizes could be provided to cover pediatric to adult and cervical to sacral. Using an adult rod/screw (or cervical plate) fixation system as a reference, the distance between rods and plates at various levels may be as follows:

| | |
|---|---|
| S1 | 60 mm |
| L3 | 50 mm |
| T12 | 40 mm |
| T2-T9 | 35-40 mm |
| Cspine | 30-35 mm |

Accordingly, three appropriate sizes might be as follows, assuming some excess to be trimmed; a preferred width would be in the range of 2.5-10 mm:
Small (Adult Cervical; Child C-T): 30-35 mm+25-50 mm per arm to wrap around the instrumentation=55-85 mm;
Medium (Adult Thoracic; Child T-L): 40-45 mm+25-50 mm per arm to wrap around the instrumentation=65-95 mm; and
Large (Adult Lumbar): 55-60 mm+25-50 mm per arm to wrap around the instrumentation=75-95 mm;

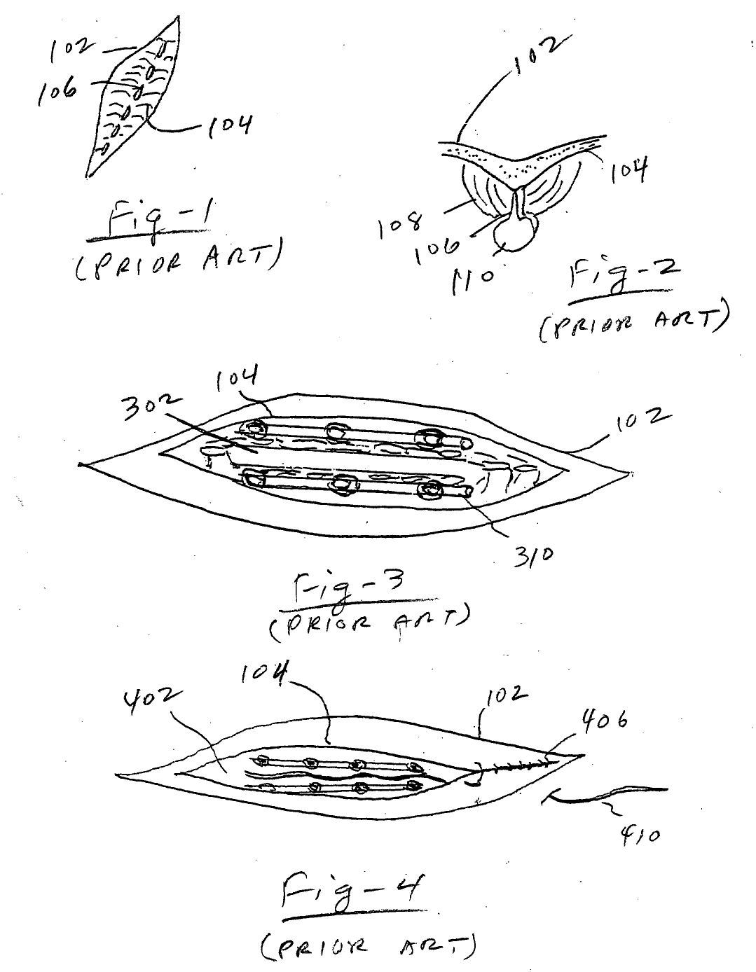

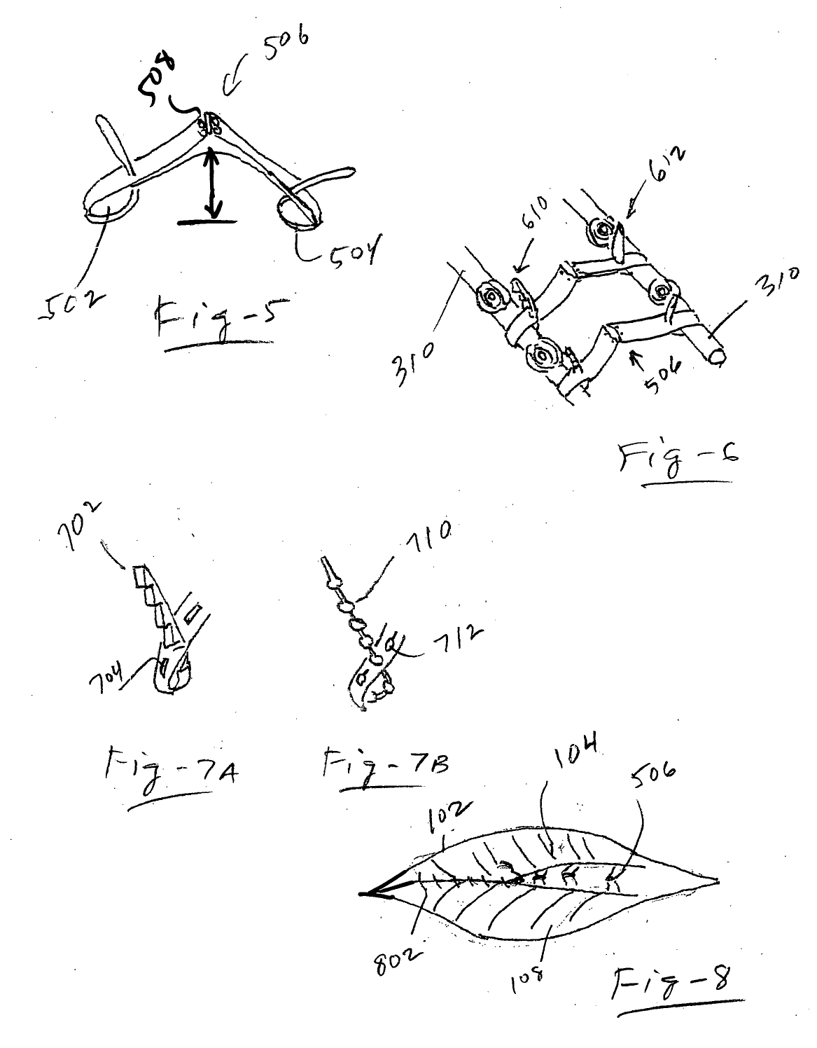

Spinous process height may be on the order of 8 mm in the L/S area to 6 mm in the cervical area. As such, for the embodiments of FIGS. 9 and 10, 4 mm(×2) may be added to the small size; 6 mm(×2) may be added for the medium size, and 8 mm(×2) may be added to the large size.

In terms of surgical procedure, the following steps may be taken according to the invention:
1. Select the number and size(s) of the anchors needed depending upon patient size and spinal level;
2. Create a spinous process if the embodiment of FIGS. 10 and 10 is being used;
3. Attach one end loosely at slot mid-point, for example;
4. Attach the other end loosely; and
5. Cinch both ends as needed, perhaps on an alternating basis to center or otherwise adjust the placement of the attachment points forming the “artificial spinous process.”

Although the invention has been described with reference to embodiments that attach to rods in particular, deep fascia anchors according to the invention may also be used in those cases whereby the fascia is released, and the spinous process is removed in the course of a decompression, for stenosis, for example. In such cases, instrumentation and fusion may not be needed but reattachment of the fascia would nevertheless be desirable. In these situations, the deep fascia anchor could be rivoted, screwed or otherwise anchored or ‘tacked down’ to the existing laminar bone. The invention is also applicable to non-spinal applications. For example, as a device that attaches to vertebra internally as an anchor for the aorta, the kidney, and other organs and blood vessels.

1. Deep fascia closure apparatus, comprising:
a device having at least one end suitable for attachment to rods, plates, or other instrumentation; and
an area to which deep fascia may be attached.

2. The apparatus of claim 1, wherein the device has two opposing arms suitable for attachment to rods, plates, or other instrumentation.

3. The apparatus of claim 2, wherein one or both of the opposing arms includes a flexible cable-tie type of connector.

4. The apparatus of claim 1, wherein the area to which deep fascia is attached includes a plurality of suture-receiving holes.

5. The apparatus of claim 1, wherein the device is provided in different physical configurations in accordance with spinal level.

6. The apparatus of claim 1, wherein the device is provided in different physical configurations in accordance with size or age of patient.
